(19) United States
(12) Patent Application Publication
(10) Pub. No.: US 2008/0075696 A1
(43) Pub. Date: Mar. 27, 2008

(54) Treatment Of Hepatitis C In The Asian Population With Interferon-Beta

(76) Inventors: Ian Parsons, Coppet (CH); Theodor Wee Tit Gin, Singapore (SG)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/792,905

(22) PCT Filed: Dec. 14, 2005
(86) PCT No.: PCT/EP05/56793

§ 371 Date: Aug. 22, 2007

Related U.S. Application Data
(60) Provisional application No. 60/637,874, filed on Dec. 20, 2004.

(30) Foreign Application Priority Data
Dec. 16, 2004 (EP) 04106652.3

(51) Int. Cl. A61K 38/21; A61K 31/41; A61K 39/395; A61P 1/16

(52) U.S. Cl. 424/85.6; 424/130.1; 514/383

(57) ABSTRACT

The invention relates to the use of IFN-beta in the manufacture of a medicament for the treatment of an HCV infection in a treatment-naïve patient belonging to the Asian population.

### FIELD OF THE INVENTION

This invention is in the field of viral diseases. More specifically, it relates to the use of IFN-beta for the manufacture of a medicament for the treatment of an HCV infection in a patient of the Asian population who has not received previous treatment with interferon-alpha.

### BACKGROUND OF THE INVENTION

Hepatitis viruses are a family of viruses comprising at present Hepatitis A virus, Hepatitis B virus, Hepatitis C virus, Hepatitis D virus, Hepatitis G virus and Hepatitis E virus.

HCV causes at least 80% of post-transfusion hepatitis cases and a substantial proportion of sporadic acute hepatitis cases. It is also implicated in many cases of chronic hepatitis, cryptogenic cirrhosis, and hepatocellular carcinoma unrelated to HBV. Infection is most commonly acquired via blood, either from transfusion or intravenous drug use. A small proportion of seemingly healthy persons are chronic HCV carriers, who often have sub-clinical chronic hepatitis or even cirrhosis. The prevalence varies with geography and other epidemiologic factors, including previous use of illicit drugs.

HCV is associated with essential mixed cryoglobulinemia, porphyria cutanea tarda, about 60 to 80% of porphyria patients have HCV, but only a few HCV patients develop porphyria. Glomerulonephritis and other “immune” disorders may be associated with the HCV infection as well. In addition, up to 25% of patients with alcoholic liver disease also harbor HCV. The reasons for this surprisingly frequent association are unclear, because concomitant alcohol and drug abuse accounts for only a portion of cases. HCV may act synergistically to exacerbate alcohol-induced liver damage, and vice versa (source: http://www.merck.com/mrkshared/mmanual/section4/chapter42/42b.jsp).

Among the hepatitis viruses, hepatitis C has the highest likelihood of chronicity, up to 75 to 80%, even though the initial illness usually seems mild. The resultant chronic hepatitis is usually benign and sub-clinical, but cirrhosis eventually develops in at least 20% of patients and this may take decades to appear. Hepatocellular carcinoma is a risk in HCV-induced cirrhosis, although tumors appear only rarely in non-cirrhotic cases of chronic infection.

Hepatitis C virus (HCV) is now known to cause most cases of what was previously termed non-A, non-B (NANB) hepatitis. This single-stranded, flavivirus-like RNA virus causes the vast majority of post-transfusion and sporadic NANB hepatitis. Multiple HCV subtypes exist with varying amino acid sequences (genotypes), and these subtypes vary geographically and play a role in disease virulence. There are 6 major HCV genotypes (Simmonds, 1999). The specific genotype does not predict the outcome of infection, it does predict the likelihood of treatment response and in many cases determines the duration of treatment (Strader et al., 2004).

HCV genotyping can be done by direct sequence analysis, by reverse hybridization to genotype-specific oligonucleotides probes, or by the use of restriction fragment length polymorphism. Tests are available for clinical use, such as the Trugene HCV 5'NC Genotyping Kit (Visible Genetics, Toronto, Canada), which is based on direct sequencing followed by comparison with a reference sequence database, and the line-probe assay (Inno LiPA HCVII, Innogenetics, Ghent, Belgium), which is based on reverse hybridization of PCR amplicons on a nitrocellulose strip coated with genotype-specific oligonucleotide probes (Ansaldi et al., 2001; Ross et al, 2000; Stuyver et al., 1996).

HCV can also alter its amino acid pattern over time in an infected person (quasi-species), and this propensity hampers vaccine development.

As mentioned above, the hepatitis C virus (HCV) produces a state of chronic infection in nearly all acutely infected individuals. Approximately 20% of patients with chronic HCV infection (CHC) develop cirrhosis with subsequent liver failure, portal hypertension, ascites, encephalopathy, and bleeding disorders (Alter M., 1992). Long-term follow-up suggests that these estimates may be conservative (Davis, 1990); moreover, chronic HCV infection is strongly associated with hepatocellular carcinoma (Tabor E. et al., 1992).

Chronic hepatitis C is presently treated with a combination of interferon-α3 million IU subcutaneous three times weekly plus oral ribavirin 1200 mg daily in two divided doses. This initially suppresses inflammation in about two third of the patients. Responders are treated for either 6 or 12 months depending on the specific viral genotype, but most relapse when treatment is stopped. Successful long-term disease suppression is only about 30-40% overall. Response depends in part on the viral genotype, viral load, and histological stage of the disease. Pegylated interferon-α, a recently developed modification of the drug molecule, may replace standard interferon-alpha in the near future because it requires injection only once a week.

Ribavirin, 1-D-ribofuranosyl-IH-1,2,4-triazole-3-carboxamide, available from ICN Pharmaceuticals, Inc., Costa Mesa, Calif., is described in the Merck Index, compound No. SI99, Eleventh Edition. Its manufacture and formulation is described in U.S. Pat. No. 4,211,771. The invention also contemplates use of derivatives of ribavirin (see, e.g., U.S. Pat. No. 6,277,830.

Interferons (IFNs) are glycoproteins produced by the body in response to a viral infection. They inhibit the multiplication of viruses in protected cells. Interferons are species-specific, i.e. IFN produced by one species will only stimulate antiviral activity in cells of the same or a closely related species. IFNs were the first group of cytokines to be exploited for their potential anti-tumor and antiviral activities.

The three major IFNs are referred to as IFN-alpha, IFN-beta and IFN-gamma. Such main kinds of IFNs were initially classified according to their cells of origin (leukocytes, fibroblasts or T-cells). However, it became clear that several types might be produced by one cell. Hence leukocyte IFN is now called IFN-alpha, fibroblast IFN is IFN-beta and T-cell IFN is IFN-gamma. There is also a fourth type of IFN, lymphoblastoid IFN, produced in the “Namalwa” cell line (derived from Burkitt's lymphoma), which seems to produce a mixture of both leukocyte and fibroblast IFN.

Human fibroblast interferon (IFN-beta) has antiviral activity and can also stimulate natural killer cells against neoplastic cells. It is a polypeptide of about 20,000 Da induced by viruses and double-stranded RNAs. From the nucleotide sequence of the gene for fibroblast interferon, cloned by recombinant DNA technology, Derynk et al., 1980 deduced the complete amino acid sequence of the protein. It is 166 amino acid long.

Shepard et al., 1981 described a mutation at base 842 (Cys→Tyr at position 141) that abolished its anti-viral activity, and a variant clone with a deletion of nucleotides 1119-1121.

Mark et al. 1984 inserted an artificial mutation by replacing base 469 (T) with (A) causing an amino acid switch from Cys→Ser at position 17. The resulting IFN-beta was reported to be as active as the ‘native’ IFN-β and stable during long-term storage (−70° C.).

Rebif® (recombinant human Interferon-beta-1a) is a drug used for multiple sclerosis (MS) treatment. Rebif® is interferon (IFN)-beta-1a, produced by mammalian cell lines.

There is no completely effective therapy for chronic hepatitis C. As mentioned above, the best results have been obtained with interferon-alpha so far. Many clinicians only observe patients with chronic hepatitis C because of the uncertain natural history of HCV infection and the toxicity associated with interferon-alpha.

Most patients with chronic hepatitis C do not achieve complete responses to treatment with interferon-alpha. Controlled trials of interferon-alpha administered for six months resulted in normalization of serum alanine aminotransferase (ALT) in 40 to 50% of patients at the end of treatment, but this response was sustained in only 15 to 25% (Hoofnagle J H et al., 1997).

Dose escalations and increased duration of therapy have resulted in small increases in sustained response, but at the cost of increased expense and toxicity (Poynard T. et al., 1996). In addition, the benefit of higher doses is often transient and relapses are common after therapy has been discontinued (Lindsay K L et al., 1996).

A study of 35 non-responders to interferon-alpha reported no benefit from prolongation of therapy from six to 12 months, increasing the dose of interferon-alpha, switching therapy from recombinant to lymphoblastoid interferon or using steroids (Piccinino F et al., 1993).

The natural history of HCV infection following lack of response to interferon-alpha has not been adequately studied, but in one study follow-up of 28 patients for at least 2 years after therapy found only one case of eventual remission at 16 months (normalization of ALT and disappearance of HCV RNA) (Takeda T et al., 1993).

Several factors have been found to be associated with greater probability of long-term sustained response to interferon-alpha: non-type 1 genotype, low serum HCV RNA concentration, shorter duration of infection, lower body weight, mild activity on liver biopsy, absence of cirrhosis and low levels of serum ferritin, iron, transferrin saturation and hepatic iron concentration (Schvarcz R et al., 1989, Bacon B R et al., 1995, Conjeevaram H S et al., 1995, Bonkovsky H L et al., 1997).

WO 03/101478 discloses that patients with chronic hepatitis C who fail to achieve a sustained response after interferon-alpha therapy may be treated with interferon-beta.

In addition to this, results have been reported for interferon-beta therapy of acute HCV infection, with 7 of 11 patients achieving sustained normalization of ALT at one year compared to only one of 14 controls (Omata M et al., 1991).

In Japan natural IFN-beta has been used for the treatment of chronic hepatitis C and the recommended regimen is a daily dose of 3-6 MIU administered i.v. for 6-8 weeks (see Habersetzer et al. 2000).

Very poor clinical efficacy of intramuscular administration of IFN-beta (3 MU three times per week (tiw)) in HCV patients of non-Asian race has been shown (Perez R. et al., 1995).

In non-Asian (Caucasian) HCV patients subcutaneous administration (9 or 12 MU) of recombinant IFN-beta has shown efficacy at least in a group of patients (Habersetzer et al., 2000).

Kishiara et al. (1995) disclose a treatment with natural IFN-beta administered intravenously at a dose of 6 MIU to HCV patients not responding to IFN-alpha.

### SUMMARY OF THE INVENTION

It is the object of the present invention to use IFN-beta for the manufacture of a medicament for the treatment of HCV infection in a patient of the Asian population, who did not receive a previous treatment with interferon-alpha.

It is a further object of the present invention to use IFN-beta in combination with ribarvirin in a patient of the Asian population, who did not receive a previous treatment with interferon-alpha.

### DETAILED DESCRIPTION OF THE INVENTION

The present invention is based on a clinical trial showing that IFN-beta has a beneficial effect in patients of the Asian population who were not treated with interferon-alpha before.

Therefore, in accordance with the present invention, IFN-beta, or a variant/mutein, functional derivative, active fragment or fusion protein thereof having IFN-beta activity, is used in the manufacture of a medicament for the treatment of an hepatitis C virus (HCV) infection in a patient of the Asian population who has not been treated with interferon-alpha before.

The patient to be treated in accordance with the present invention has not received pre-treatment with interferon-alpha, which is a common anti-HCV treatment. Such a patient is also called “treatment-naïve” or “interferon-alpha-naïve” herein.

In a preferred embodiment of the invention, the patient has not received any treatment with a known anti-HCV drug (such as e.g. ribavirin). It is also preferred that the patient has not received a combination of anti-HCV drugs before receiving IFN-beta in accordance with the invention. In particular, preferably, the patient has not been treated with a combination of IFN-alpha and ribavirin before starting IFN-beta treatment in accordance with the present invention.

The term “interferon-beta (IFN-beta or IFN-β)”, as used in the present invention, is intended to include human fibroblast interferon, which may be native, i.e. purified from a natural source, or obtained by DNA recombinant techniques from prokaryotic sources (e.g. Escherichia coli, E. coli) or from eukaryotic host cells, e.g. from yeast or mammalian cells. Mammalian cells such as Chinese hamster ovary cells (CHO) or human cells are a preferred host for production of recombinant IFN-beta. The IFN-beta may be glycosylated or non-glycosylated. The term “interferon-beta”, as used herein, encompasses natural interferon-beta as well as interferon-beta produced by recombinant means, be it from prokaryotic (e.g. E. coli) or eukaryotic (e.g. CHO) hosts. If IFN-beta, used in accordance with the present invention, is non-glycosylated (e.g. produced in E. coli), it is preferred to administer higher amounts of IFN-beta in order to obtain a biological or pharmacological effect comparable to that of glycosylated IFN-beta. For instance, an amount of non-glycosylated IFN-beta that is about 10 times higher than the amount of glycosylated IFN-beta is preferably administered in order to obtain comparable activities. The term “interferon-beta”, as used herein, also encompasses functional derivatives, muteins, analogs, and fragments, or fusion proteins of IFN-beta.

Preferably, the IFN-beta to be used in the frame of the present invention is Avonex®, Betaseron®, or, more preferably, Rebif®.

Rebif® (interferon beta-1a) is a purified 166 amino acid glycoprotein with a molecular weight of approximately 22,500 daltons. It is produced by recombinant DNA technology using genetically engineered Chinese Hamster Ovary cells into which the human interferon beta gene has been introduced. The amino acid sequence of Rebif® is identical to that of natural fibroblast derived human interferon beta. Natural interferon beta and interferon beta-1a (Rebif®) are glycosylated with each containing a single N-linked complex carbohydrate moiety.

Using a reference standard calibrated against the World Health Organization natural interferon beta standard (Second International Standard for Interferon, Human Fibroblast GB 23 902 531), Rebif® has a specific activity of approximately 270 million international units (MIU) of antiviral activity per mg of interferon beta-1a determined in an in vitro cytopathic effect bioassay using WISH cells and Vesicular Stomatitis virus.

Conversion table for MIU and mcg of IFN-beta

| | | | | |
|---|---|---|---|---|
| MIU | 3 | 12 | 18 | 24 |
| mcg | 11 | 44 | 66 | 88 |

Rebif® 44 mcg contains approximately 12 MIU of antiviral activity using this method.

“Functional derivatives” as used herein cover derivatives of IFN-beta, and its variants or muteins and fused proteins, which may be prepared from the functional groups which occur as side chains on the residues or the N- or C-terminal groups, by means known in the art. These functional derivatives are included in the invention as long as they remain pharmaceutically acceptable, i.e. they do not destroy the activity of the protein, which is substantially similar to, or better than, the activity of IFN-beta, and do not confer toxic properties on compositions containing it.

These derivatives may, for example, include polyethylene glycol side-chains, which may improve other properties of the protein, such as the stability, half-life, bioavailability, tolerance by the human body, or immunogenicity. To achieve this goal, IFN-beta may be linked e.g. to Polyethlyenglycol (PEG). PEGylation may be carried out by known methods, described in WO 92/13095, for example. In particular, PEG-IFN can be prepared in accordance with the teaching of WO 99/55377.

Therefore, in a preferred embodiment, the functional derivative of IFN-beta comprises at least one moiety attached to one or more functional groups, which occur as one or more side chains on the amino acid residues. An embodiment in which the moiety is a polyethylene glycol (PEG) moiety is highly preferred. In accordance with the present invention, several PEG moieties may also be attached to the IFN-beta.

Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

“Variants” or “muteins”, as used in the frame of the present invention, refer to analogs of IFN-beta, in which one or more of the amino acid residues of natural IFN-beta are replaced by different amino acid residues, or are deleted, or one or more amino acid residues are added to the natural sequence IFN-beta, without diminishing considerably the activity of the resulting products as compared with the wild type IFN-beta. These muteins are prepared by known synthesis and/or by site-directed mutagenesis techniques, or any other known technique suitable therefor.

The terms “variant” or “mutein” in accordance with the present invention include proteins encoded by a nucleic acid, such as DNA or RNA, which hybridizes to DNA or RNA encoding IFN-beta as disclosed e.g. in U.S. Pat. No. 4,738,931 under stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992). Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotides or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called global alignment), that is particularly suitable for sequences of the same or very similar length, or over shorter, defined lengths (so-called local alignment), that is more suitable for sequences of unequal length.

Methods for comparing the identity and homology of two or more sequences are well known in the art. Thus for instance, programs available in the Wisconsin Sequence Analysis Package, version 9.1 (Devereux J et al., 1984), for example the programs BESTFIT and GAP, may be used to determine the % identity between two polynucleotides and the % identity and the % homology between two polypeptide sequences. BESTFIT uses the “local homology” algorithm of Smith and Waterman (1981) and finds the best single region of similarity between two sequences. Other programs for determining identity and/or similarity between sequences are also known in the art, for instance the BLAST family of programs (Altschul S F et al, 1990, Altschul S F et al, 1997, accessible through the home page of the NCBI at www.ncbi.nlm.nih.gov) and FASTA (Pearson W R, 1990).

Any such variant or mutein preferably has a sequence of amino acids sufficiently duplicative of that of IFN-beta, such as to have substantially similar activity to IFN-beta. A functional assay for evaluating whether any variant or mutein has a similar activity as IFN-beta is e.g. the assay measuring the activity of interferon on the cytopathic effect of vesicular stomatitis virus in WISH cells, e.g. described by Youcefi et al., 1985. Thus, it can be determined whether any given mutein has substantially the same activity as IFN-beta by means of routine experimentation.

In a preferred embodiment, any such variant or mutein has at least 40% identity or homology with the sequence of IFN-beta as disclosed e.g. in U.S. Pat. No. 4,738,931. More preferably, it has at least 50%, at least 60%, at least 70%, at least 80% or, most preferably, at least 90% identity or homology thereto.

Muteins of IFN-beta, which can be used in accordance with the present invention, or nucleic acid coding therefor, include a finite set of substantially corresponding sequences as substitution peptides or polynucleotides which can be routinely obtained by one of ordinary skill in the art, without undue experimentation, based on the teachings and guidance presented herein.

Preferred changes for muteins in accordance with the present invention are what are known as “conservative” substitutions. Conservative amino acid substitutions of IFN-beta polypeptides may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g., under thirty, and preferably under ten, and do not remove or displace amino acids which are critical to a functional conformation, e.g., cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Met |

Examples of production of amino acid substitutions in proteins which can be used for obtaining muteins of IFN-beta for use in the present invention include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al).
A special kind of interferon variant has been described recently. The so-called “consensus interferons” are non-naturally occurring variants of IFN (U.S. Pat. No. 6,013,253). Consensus interferons may also be used according to the invention.

“Functional derivatives” of IFN-beta as used herein covers derivatives which may be prepared from the functional groups which occur as side chains on the residues or the N- or C-terminal groups, by means known in the art, and are included in the invention as long as they remain pharmaceutically acceptable, i.e., they do not destroy the biological activity of the proteins as described above, i.e., the ability to bind the corresponding receptor and initiate receptor signaling, and do not confer toxic properties on compositions containing it. Derivatives may have chemical moieties, such as carbohydrate or phosphate residues, provided such a derivative retains the biological activity of the protein and remains pharmaceutically acceptable.

For example, derivatives may include aliphatic esters of the carboxyl groups, amides of the carboxyl groups by reaction with ammonia or with primary or secondary amines, N-acyl derivatives or free amino groups of the amino acid residues formed with acyl moieties (e.g., alkanoyl or carbocyclic aroyl groups) or O-acyl derivatives of free hydroxyl group (e.g., that of seryl or threonyl residues) formed with acyl moieties. Such derivatives may also include for example, polyethylene glycol side-chains, which may mask antigenic sites and extend the residence of the molecule in body fluids.

Of particular importance is a protein that has been derivatized or combined with a complexing agent to be long lasting. For example, pegylated versions, or proteins genetically engineered to exhibit long lasting activity in the body, can be used according to the present invention. A pegylated version of interferon-beta-1a has been described in WO 99/55377 and is considered as included in the definition of interferon-beta according to the present application.

In accordance with the present invention, a salt of IFN-beta may also be used for treatment of HCV infections in interferon-alpha treatment naïve patients.

The term “salts” herein refers to both salts of carboxyl groups and to acid addition salts of amino groups of the proteins described above or analogs thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of IFN-beta, which may be measured e.g. in the bioassay explained above.

The term “fused protein” refers to a polypeptide comprising IFN-beta, or a variant or mutein or fragment thereof, fused with another protein, which, e.g., has an extended residence time in body fluids. IFN-beta may thus be fused to another protein, polypeptide or the like, e.g., an immunoglobulin or a fragment thereof.

Therefore, in a further embodiment, IFN-beta comprises an immunoglobulin fusion, i.e. IFN-beta is a fused protein comprising all or part of IFN-beta fused to all or a portion of an immunoglobulin. Methods for making immunoglobulin fusion proteins are well known in the art, such as the ones described in WO 01/03737, for example. The person skilled in the art will understand that the resulting fusion protein of the invention retains the biological activity of IFN-beta. The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met, or a Gly-Ser rich linker introduced between the IFN-beta sequence and the sequence derived from an immunoglobulin sequence. The resulting fusion protein has improved properties, such as an extended residence time in body fluids (half-life), increased specific activity, increased expression level, or the purification of the fusion protein is facilitated.

In a further preferred embodiment, IFN-beta is fused to the constant region of an Ig molecule, often called the Fc part of the immunoglobulin. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM or IgA, for example. Fusion proteins may be monomeric or multimeric, hetero- or homomultimeric. Methods of preparing immunoblobulin fusion proteins are known in the art, e.g. from EP 526 452 or from U.S. Pat. No. 5,155,027. Ig fusion proteins comprising IFN-beta moieties are described e.g. in EP 227 110, U.S. Pat. No. 5,541,087, WO 97/24137 or WO 00/23472.

A “fragment” according to the present invention refers to any subset of IFN-beta, that is, a shorter peptide, which retains the desired biological activity as measurable e.g. in the bioassay described above. Fragments may readily be prepared by removing amino acids from either end of the molecule and testing the resultant for its properties as a receptor agonist. Proteases for removing one amino acid at a time from either the N-terminal or the C-terminal of a polypeptide are known, and so determining fragments, which retain the desired biological activity, may be determined e.g. in the test described by Youcefi et al., 1985, and involves only routine experimentation.

While the present invention provides recombinant methods for making the above-defined derivatives, these derivatives may also be made by conventional protein synthesis methods, which are well known to those skilled in the art.

According to the present invention patient belonging to an Asian population, or a patient belonging to the Asian race is used synonymously. A “race” or “population”, as used herein, is a population that can be distinguished as a distinct subgroup within a species (e.g. the human species). A race possesses a unique and distinct ensemble of genes, and is identified by the traits (both mental and physical) produced by the genetic ensemble. Members of the same race share distinguishing genetic characteristics, because they share a common genetic ancestry and a consequently similar genetic ensemble.

Based on the nuclear DNA studies of Luigi Cavalli Sforza and his colleagues at least 6 human races/populations can be defined: the Caucasoid (which include the European and Indian populations), the African, the Asian, the Arctic, the American Indian, and the Pacific (Cavalli-Sforza et al., 1991).

According to the present invention “Asian” means any person having origins in any of the original peoples of China, Mongolia, Taiwan, Singapore, Korea, Japan, Vietnam, Cambodia, Laos, Burma, Thailand, Malaysia, Indonesia and Philippines.

In a another embodiment an Asian subpopulation is treated, which includes any person having origins in any of the original peoples of China, Mongolia, Taiwan, Singapore, Korea, Vietnam, Cambodia, Laos, Burma, Thailand, Malaysia, Indonesia and Philippines.

In another embodiment an Asian subpopulation is treated, which includes any person having origins in any of the original peoples of China, Mongolia, Taiwan, Singapore, Korea, Vietnam, Cambodia, Laos, Burma, Thailand.

In another embodiment an Asian subpopulation is treated, which includes any person having origins in any of the original peoples of China, Mongolia, and Taiwan.

In another embodiment an Asian subpopulation is treated, which includes any person having origins in any of the original peoples of China.

In another embodiment an Asian subpopulation is treated, which includes any person having origins in any of the original peoples of Japan.

“Non-Asian” is herein intended to refer to all the other human races/populations, which do not fall under the above-definition of “Asian”. Patients normally are requested to self-identify by “race” or population by the doctor on the basis of their somatic traits and/or the country of origin assigns the race.

In a preferred embodiment of the invention, the HCV infection to be treated by IFN-beta, a mutein, active fragment, functional derivative or fusion protein thereof, in an IFN-alpha treatment-naïve patient of the Asian population, is a chronic HCV infection.

Generally, HCV infection is diagnosed by detecting HCV in the blood, e.g. by using antibodies to detect viral proteins (immunoassays such as EIA, ELISA or strip immunoassay, SIA) or, by detecting viral RNA either in a qualitative or quantitative assay.

Qualitative tests include e.g. tests based on PCR or transcription-mediated amplification (TMA), such as e.g. the commercially available Amplicor™ Hepatitis C Virus Test, or the Cobas Amplicor™ Hepatitis C Virus test (Roche Molecular Systems, Branchburg, N.J.).

Quantitative assays are based on RT PCR or “branched DNA” assays, such as the Amplicor™ HCV Monitor version #2.0, the Cobas Amplicor™ Monitor Version #3.0 quantitative assay (both Roche), the Versant™ HCV RNA version #3.0 Quantitative Assay (Bayer Diagnostics) the LCx™ HCV RNA Quantitative Assay (Abbott) or the SuperQuant™ Assay (National Genetics Institute, LA).

Hepatitis C virus to be treated in accordance with the present invention may be of any genotype, i.e. genotype 1, including genotypes 1a and 1b,2,3,4,5 or 6.

In a preferred embodiment, the HCV is selected from a genotype that is difficult to treat by currently known treatment methods, i.e. IFN-alpha monotherapy, or IFN-alpha in combination with ribavirin. Such difficult-to-treat HCV genotypes are particularly genotypes 1, 4, 5 or 6. The HCV genotype the patient has been infected with may be determined on the basis of direct HCV sequencing or reverse hybridization of PCR amplicons with genotype-specific probes, or by (RT) PCR with adequate primers.

It is particularly preferred to treat patients according to the invention that are infected with HCV of genotype 1, which is usually very difficult or even resistant to treatment (Sievert, 2002 or Strader et al., 2004).

Thus, the present invention includes the treatment of patient populations infected with HCV genotype 1a and/or HCV genotype 1b. Within HCV genotype 1b three subtypes may be distinguished, each named for its geographical prevalence: subtype W (worldwide), subtype J (Japan) and subtype NJ (Not in Japan). The present invention includes the treatment of patient populations infected with HCV genotype 1b of the which are infected with subtype W, subtype J and/or subtype NJ. In particular, the treatment of Asian populations and Asian subpopulations as herein defined which are infected with these subtypes is contemplated.

In order to evaluate, whether treatment of IFN-beta is efficient in a patient, several parameters may be measured, such as the change of viral load in terms of HCV RNA in the serum, a normalization of the ALT levels, improvement of at least two points in each component of the liver necroinflammation score (HAI) or, improvement of architectural staging (liver fibrosis) by at least one point. Grading and staging of the histopathological lesions of chronic hepatitis are described by Brunt (2000).

The present invention also includes the treatment of HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-cc-based therapies or who cannot tolerate IFN—based therapies, or who have a contraindication to such therapies. In particular embodiments of interest, HCV-positive individuals with stage 3 or 4 liver fibrosis according to the METAVIR scoring system are suitable for treatment with the methods of the present invention. In other embodiments, individuals suitable for treatment with the methods of the instant invention are patients with decompensated cirrhosis with clinical manifestations, including patients with far-advanced liver cirrhosis, including those awaiting liver transplantation. In still other embodiments, individuals suitable for treatment with the methods of the instant invention include patients with milder degrees of fibrosis including those with early fibrosis (stages 1 and 2 in the METAVIR, Ludwig, and Scheuer scoring systems; or stages 1, 2, or 3 in the Ishak scoring system).

Measurement of ALT may be carried out e.g. by the in vitro assay for the quantitative determination of alanine aminotransferase with pyrodoxal phosphate activation in human serum and plasma on automated chemistry analyzers as commercially available e.g. as the ALT (ALAT/GPT) method (Roche Diagnostics).

The patients that are to be treated in accordance with the present invention may have a high virus load, such as e.g. a viral load of ≧10⁴ or ≧10⁵ or ≧10⁶ copies/ml serum. Viral load is measured e.g. using the quantitative assays described above.

In a preferred embodiment, the patient has a high viral load of over 2×10⁶ copies/ml.

The patient treated in accordance with the present invention may have an early virologic response, EVR, or a sustained viral response, SVR.

“EVR”, as used herein, is defined as a ≧log decline or absence of HCV RNA from baseline after 12 weeks of treatment.

“SVR”, as used herein, is defined as the absence of detectable HCV RNA in the serum both at the end of treatment and at the end of 24 weeks of observation period (during which there is no treatment). The treatment may be either 24 or 48 weeks.

SVR is currently the standard to determine whether a patient is “cured” from HCV infection. From studies with IFN-alpha and ribavirin it is known that EVR is highly predictive for SVR (Strader et al., 2004, Davis et al., 2003).

In a further preferred embodiment, the patient has a sustained viral response. “Sustained viral response”, as used herein, is defined as the absence of detectable HCV RNA in the serum both after about 12 or about 16 or about 20 or about 24 or about 28 or about 32 or about 36 or about 40 or about 44 or about 48 weeks of treatment.

IFN-beta, or a variant/mutein, functional derivative, active fragment or fusion protein thereof having IFN-beta activity, is preferably administered systemically, and preferably subcutaneously or intramuscularly. Intradermal, transdermal (e.g. in slow release formulations), intravenous, oral, intracranial, epidural, topical, rectal, and intranasal routes are also within the present invention.

Any other therapeutically efficacious route of administration may also be used, for example absorption through epithelial or endothelial tissues or by gene therapy wherein a DNA molecule encoding the IFN-beta is administered to the patient (e.g. via a vector), which causes IFN-beta to be expressed and secreted in vivo.

IFN-beta may be formulated as a pharmaceutical composition, i.e. together with a pharmaceutically acceptable carrier, excipients or the like.

The definition of “pharmaceutically acceptable” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

A “therapeutically effective amount” of IFN-beta is such that when administered, IFN-beta exerts an anti-HCV effect.

The dosage administered, as single or multiple doses, to an individual will vary depending upon a variety of factors, including IFN-beta pharmacokinetic properties, the route of administration, patient conditions and characteristics (sex, age, body weight, health, size), extent of symptoms, concurrent treatments, frequency of treatment, and HCV genotype the patient is infected with. Adjustment and manipulation of established dosage ranges may be determined by those skilled in the art.

Preferred doses and regimens in accordance with the present invention are selected from the group consisting of: 12 MIU (44 mcg) of IFN-beta three times a week, 12 MIU (44 mcg) daily, 24 MIU (88 mcg) three times a week, 24 MIU (88 mcg) daily. These doses are preferably administered subcutaneously.

It is also preferred to administer IFN-beta at 100 mcg (about 27 MIU) once per week intramuscularly.

In one embodiment IFN-beta is administered for at least 8 weeks or at least 12 weeks.

In another embodiment IFN-beta is administered for no longer than 48 weeks, or no longer than 36 weeks, or no longer than 24 weeks.

In a preferred embodiment IFN-beta is administered for about 24 weeks, such as for example a treatment schedule for 24 weeks.

These schedules are particularly preferred when IFN-beta is given subcutaneously, in particular when recombinant IFN-beta is given subcutaneously, such as in particular CHO-derived recombinant IFN-beta 1a.

The daily doses may also be given in divided doses or in sustained release form effective to obtain the desired results. Second or subsequent administrations can be performed at a dosage which is the same, less than or greater than the initial or previous dose administered to the individual.

According to a further preferred embodiment of the present invention the treatment with IFN-beta can be coupled with another antiviral drug. A commonly used antiviral drug in the treatment of HCV is ribavirin (a nucleoside analog), but other drugs show some potential in this treatment and are listed in e.g. in Wilkinson, 2002) and include serine protease inhibitors, inhibitors of the RNA-dependent RNA polymerase (RdRp) and helicase inhibitors. These drugs can be administered simultaneously, separately or sequentially with recombinant IFN-beta. Such antiviral drugs may also be chosen from interferons other than IFN-beta, such as for example IFN-alpha and IFN-omega. Therefore, in a highly preferred embodiment, IFN-beta is used in combination with ribavirin. The IFN-beta and ribavirin may be used separately, sequentially or simultaneously. Ribavirin may preferably be administered orally, and preferably at about 800 or about 900 or about 1000 or about 1100 or about 1200 or about 1300 or about 1400 mg per person per day, e.g. in a single does or in two separate doses. It is highly preferred to administer e.g. about 1000 mg per day for a patient weighing less than about 75 kg and about 1200 mg per day for patients weighing more than about 75 kg.

In a further aspect, the present invention relates to a method for treating an HCV infection comprising administering an effective amount of IFN-beta, or a Varaint/mutein, functional derivative, active fragment or fusion protein thereof having IFN-beta activity, together with a pharmaceutically acceptable excipient, to a patient of the Asian population who has not been treated with an interferon before.

Having now fully described this invention, it will be appreciated by those skilled in the art that the same can be performed within a wide range of equivalent parameters, concentrations and conditions without departing from the spirit and scope of the invention and without undue experimentation.

While this invention has been described in connection with specific embodiments thereof, it will be understood that it is capable of further modifications. This application is intended to cover any variations, uses or adaptations of the invention following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning an range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### EXAMPLE 1

### Early Virologic Response of Interferon-Beta-1a (Rebif®) Therapy in Asian Patients with Hepatitis C: 12 Weeks Results from a Multicentre, Randomized, Placebo-Controlled Trial

Objectives

The primary objective of the study was to evaluate and compare the effect of interferon-beta-1a to placebo on sustained virologic response (SVR) in Asian patients with IFN-alpha treatment-naïve chronic hepatitis C infection.

The main secondary objective of the study was to evaluate and compare the effect of interferon-beta-1a to interferon-beta-1a/ribavirin combination on SVR.

The other secondary objectives of the study were to evaluate and compare interferon-beta-1a to a) placebo and b) interferon-beta-1a/ribavirin combination for the effect of treatment on viral load and clearance, alanine aminotransferase (ALT) levels, liver histology, and the safety and tolerability of treatment, including haematological and biochemical parameters and the development of neutralizing antibodies.

The current example provides the results from an analysis on the response after the first 12 weeks of treatment between the interferon-beta-1a group and the placebo group.

The sustained viral response, SVR, will be evaluated at the end of the 24 weeks treatment period.

Materials and Methods

Patient Selection

Eligible subjects were Asian patients who had never received interferon and had the following characteristics: age greater than 18 years, ALT levels between 1.5 times and 10 times the upper limit of normal on two occasions, at least four weeks apart from each other, within a period of at least six months and at screening visit, and hepatitis C infection demonstrated by HCV-RNA assay or PCR, and a liver biopsy (within 1 year) showing histological features consistent with chronic hepatitis C.

Criteria for exclusion were neutropenia (neutrophil count, <1500 per cubic millimeter); thrombocytopenia (plate count, <100,000 per cubic millimeter); a creatinine concentration above the upper limit of normal; a serum alpha-fetoprotein concentration above 100 μg per liter; history of hepatic failure or immunologically mediated disease or severe retinopathy, chronic renal impairment, positive test for HBsAg, IgM anti-HBc or anti-HIV, clinical evidence of liver cancer, or known and ongoing alcohol or drug abuse.

Study Design

This phase III, multicenter, randomized, partially-blinded, placebo-controlled study was conducted in 16 investigational centers at 3 countries—China, Hong Kong and Singapore between August 2002 and June 2004. Patients were equally randomized to 12 weeks of blinded treatment of either interferon-beta-1a (Serono, Rebif 44 mcg subcutaneously (sc, three times per week (tiw)) or placebo. The treatments were unblinded at week 12. Patients in the interferon-beta-1a group continued with another 12 weeks of interferon-beta-1a treatment followed by 24 weeks of observation period. Patients in the placebo group who did not have viral clearance at week 12 were treated with a combination of interferon-beta-1a (Serono, Rebif 44 mcg subcutaneous (sc) three times per week (tiw)) and ribavirin (orally, 1000 mg per day for patients weighing ≦75 kg and 1200 mg per day for patients >75 kg) for 24 weeks followed by 24 weeks of observation period.

SVR was defined as the absence of detectable HCV RNA in the serum both at the end of 24 weeks of treatment period and at the end of 24 weeks of observation period.

EVR (Early Virologic Response) was defined as a reduction in HCV RNA by at least 2 logs after the first 12 weeks of treatment compared with baseline.

The institutional review boards of the participating centers approved the protocol, and all patients provided written informed consent. The study was conducted according to the guidelines of the Declaration of Helsinki, and the applicable provision of Good Clinical Practices.

Virology

All virologic assessments were performed in a central laboratory. At baseline, genotypes were identified using a line probe hybridization assay, the Versant™ HCV Genotype Amplification Kit (LIPA™), available from Bayer under Ref. 128448, according to the Manufacturer's protocol. HCV RNA was quantified by the branched DNA signal amplification assay (Versant™ 3.0). HCV RNA clearance was determined by the more sensitive COBAS-AMPLICOR™ assay (Amplicor 2.0, Roche, Pleasanton, Calif.). Both the Versant 3.0 assay and COBAS AMPLICOR assay were used to test viremia in each patient at the scheduled visit during the study. SVR was determined using the results from the COBAS-AMPLICOR assay.

Measurement of ALT was done using the in vitro assay for the quantitative determination of alanine aminotransferase with pyrodoxal phosphate activation, available as the ALT (ALAT/GPT) standard method 94 (Roche Diagnostics on a Roche/Hitachi 917 instrument).

Assessment of Efficacy

The main analysis of the primary efficacy endpoint to test the effect of treatment on SVR will be performed using logistic regression, adjusted for baseline HCV RNA, genotype and center/country. The treatment groups for the main comparison of SVR include the patients randomized to interferon-beta-1a versus those randomized to placebo.

Unadjusted logistic regression will be performed as supportive analysis. Adjusted and unadjusted odds ratios and the two-sided 95% confidence interval will be obtained. Secondary efficacy endpoints that involve proportions at a time point will be analyzed similarly to the primary efficacy endpoint. Secondary efficacy endpoints that involve change from baseline for continuous variables will be analyzed using linear mixed models.

Assessment of Safety

Adverse events were graded according to the modified NCl CTC toxicity criteria as published by the National Cancer Institute under the title “COMMON TOXICITY CRITERIA MANUAL—Common Toxicity Criteria, Version 2.0” dated Jun. 1, 1999, available e.g. underwww.rtog.org/members/toxicity/ctcmanual6-1-99.pdf.

If an adverse event occurs that is not listed among these criteria, the Investigator evaluated its severity as mild, moderate, severe, or very severe, and their occurrence was assessed throughout the treatment period and for 24 weeks after the end of treatment.

Dose reduction of ribavirin for selected haematological parameters were allowed to manage adverse events or laboratory abnormalities that had reached predetermined thresholds of severity. The Investigator was allowed to interrupt the treatment for up to two weeks for any medical and/or safety reason without discontinuing the patient from the study. Patients who discontinued their assigned treatment were encouraged to remain in the study for assessment at the Week 48 visit.

Statistical Analysis

There were three planned analyses: preliminary, primary and final analyses.

The preliminary analysis was conducted as soon as all patients complete the double-blind treatment phase (i.e. after 12 weeks of treatment) on the following endpoints:
proportion of patients with complete viral clearance at week 12 of Treatment Period;
proportion of patients with ALT normalization at week 12 of the Treatment Period; and
change in viral load from baseline to week 12 of Treatment Period.

The primary analysis will be conducted as soon as all of the interferon-beta-1a patients have completed the study. This analysis will be performed on patients who have HCV RNA values both at study weeks 24 and 48 for the patients randomized to interferon-beta-1a. If a patient does not have a Week 48 assessment, then the last non-missing post-baseline assessment will be carried forward. Those patients randomized to placebo, who are non-responders at week 12 will be classed as non-SVR. Those who are responders at week 12 will be classed as SVR, unless data is available at week 24 and 48 data for the responders which suggests otherwise (see table below for further details)

| Treatment | | |
|---|---|---|
| Group | SVR | Non-SVR |
| Placebo | HCV clearance at the end | No HCV clearance at the |
| | of placebo treatment | end of placebo treatment |
| | (week 12) and clearance | (week 12) or at the time |
| | at both end of Treatment | of withdrawal from placebo. |
| | Period (week 24) and | |
| | Observation Period | |
| | (week 48) or at the time | |
| | of withdrawal from placebo | |
| | (natural responder). | |
| Interferon- | HCV clearance both at | No HCV clearance at the |
| beta-1a | the end of Treatment | end of the Treatment or |
| | Period and end of | Observation Period or |
| | Observation Period. | at the time of withdrawal. |

The final analysis will be carried out once all patients complete the study. All data for the interferon-beta-1a group and the placebo responders will be included in these analyses plus data from the first dose of interferon-beta-1a to the end of the observation period for the placebo non-responders (i.e. those patients who crossed over to receive interferon-beta-1a and ribavirin in combination).

Results

Patient Demographics

Of the 406 patients screened, 257 met the criteria for entry and underwent randomization. 128 patients were randomly assigned to interferon-beta-1a treatment group and 129 were in placebo group. The baseline characteristic of the patients enrolled in the two treatment groups were similar (Table 1 and 2). Of the 257 patients enrolled, 255 completed the 12 weeks double-blind phase of the study. Overall, 65% of patients were infected with HCV genotype 1 and 65% of patients had high pre-treatment viral load (≧2×10⁶ copies/ml).

TABLE 1
Summary of demographic and baseline characteristics

| | IFN-beta-1a | | Placebo | | Overall | |
|---|---|---|---|---|---|---|
| | N = 128 | | N = 129 | | N = 257 | |
| Sex | | | | | | |
| Male | 92 | (71.9%) | 89 | (69.0%) | 181 | (70.4%) |
| Female | 36 | (28.1%) | 40 | (31.0%) | 76 | (29.6%) |
| Age (years) | | | | | | |
| Mean (SD) | 38.5 | (11.46) | 40.2 | (10.91) | 39.3 | (11.20) |
| Min/Max | 18/65 | | 19/62 | | 18/65 | |
| Race | | | | | | |
| Asian | 128 | (100.0%) | 129 | (100.0%) | 257 | (100.0%) |
| Black | 0 | | 0 | | 0 | |
| White | 0 | | 0 | | 0 | |
| Other | 0 | | 0 | | 0 | |
| Height (cm) | | | | | | |
| Mean (SD) | 168.3 | (7.85) | 168.0 | (7.32) | 168.2 | (7.57) |
| Min/Max | 140/182 | | 150/185 | | 140/185 | |
| Weight (kg) | | | | | | |
| Mean (SD) | 68.3 | (11.86) | 64.9 | (10.45) | 66.6 | (11.28) |
| Min/Max | 42/104 | | 43/95 | | 42/104 | |
| BMI (kg/m²) | | | | | | |
| Mean (SD) | 24.02 | (3.215) | 22.95 | (3.062) | 23.49 | (3.179) |

| | | | |
|---|---|---|---|
| Min/Max | 16.4/33.9 | 16.0/30.9 | 16.0/33.9 |

BMI = Body Mass Index;
IFN = interferon;
N = number of subjects in Full Analysis Set;
SD = standard deviation.

TABLE 2
Summary of baseline disease characteristics

| | IFN-beta-1a | | Placebo | | Overall | |
|---|---|---|---|---|---|---|
| | N = 128 | | N = 129 | | N = 257 | |
| Viral load (copies/mL) | | | | | | |
| Mean (SD) | 6,240,819 | (7,311,177) | 5,573,984 | (6,791,989) | 5,906,104 | (7,049,468) |
| Min/Max | 6600/56,451,420 | | 6600/50,989,430 | | 6600/56,451,420 | |
| Viral load^{A} | | | | | | |
| Low | 43 | (33.6%) | 47 | (36.4%) | 90 | (35.0%) |
| High | 85 | (66.4%) | 82 | (63.6%) | 167 | (65.0%) |
| HCV genotype | | | | | | |
| Type-1 | 85 | (66.4%) | 82 | (63.6%) | 167 | (65.0%) |
| Type-2 | 23 | (18.0%) | 27 | (20.9%) | 50 | (19.5%) |
| Type-3 | 10 | (7.8%) | 11 | (8.5%) | 21 | (8.2%) |
| Type-4 | 1 | (0.8%) | 0 | | 1 | (0.4%) |
| Type-5 | 0 | | 0 | | 0 | |
| Type-6 | 8 | (6.3%) | 9 | (7.0%) | 17 | (6.6%) |
| Other | 1 | (0.8%) | 0 | | 1 | (0.4%) |
| ALT level (U/L) | | | | | | |
| Mean (SD) | 132.3 | (73.29) | 136.0 | (93.58) | 134.2 | (83.95) |
| Min/Max | 23/380 | | 15/581 | | 15/581 | |
| Liver cirrhosis^{B} | | | | | | |
| Yes | 5 | (3.9%) | 6 | (4.7%) | 11 | (4.3%) |
| No | 123 | (96.1%) | 123 | (95.3%) | 246 | (95.7%) |

ALT = alanine aminotransferase;
HCV = hepatitis C virus;
IFN = interferon;
N = Number of subjects in Full Analysis set;
SD = standard deviation
^{A}Low viral load = ≧2,000,000 copies/mL; High viral load = >2,000,000 copies/mL
^{B}“Does the pre-study liver biopsy reveal probable or definite cirrhosis?”

Early Virologic Response (EVR)

By week 12, rates of EVR, complete viral clearance, and ALT normalization, were significantly higher for interferon-beta-1a group (92.2%, 74.2%, 65.1%) than for placebo group (2.3%, 0%, 8.5%), respectively (Table 3, 4 and 5).

TABLE 3
Complete viral clearance at Week 12

| | IFN-beta-1a | Placebo | Overall |
|---|---|---|---|
| | N = 128 | N = 129 | N = 257 |
| Was viral clearance | | | |
| achieved? | | | |
| Yes | 95 (75.4%) | 0 | 95 (37.4%) |
| No | 31 (24.6%) | 128 (100.0%) | 159 (62.6%) |

| | | |
|---|---|---|
| Comparison | | |
| IFN-beta-1a vs placebo | | |
| Odds ratio | N/A | |
| 95% confidence interval | N/A | |
| p-value | <0.001 | <0.001 |

TABLE 4
Complete viral clearance at Week 12 or at least a 2 log
decrease in HCV RNA from baseline to Week 12 (EVR)

| | IFN-beta-1a | Placebo | Overall |
|---|---|---|---|
| | N = 128 | N = 129 | N = 257 |
| Was viral clearance | | | |
| achieved? | | | |
| Yes | 116 (92.1%) | 3 (2.3%) | 119 (46.9%) |
| No | 10 (7.9%) | 125 (97.7%) | 135 (53.1%) |

| | | |
|---|---|---|
| Comparison | | |
| IFN-beta-1a vs placebo | | |
| Odds ratio | 608.09 | |
| 95% confidence interval | (144.452, 2559.877) | |
| p-value | <0.001 | <0.001 |

TABLE 4.1
Genotype Analysis

| Complete viral clearance | | | | |
|---|---|---|---|---|
| or ≧2 log decrease in | Interferon-beta-1a | | Placebo | P- |
| HCV RNA (n, %) | (n = 128) | | (n = 129) | value |
| HCV Genotype 1 | 75/84 | (89.3%) | 2/81 (2.5%) | |
| HCV Genotype 2 | 23/23 | (100.0%) | 1/27 (3.7%) | |
| HCV Genotype 3 | 8/9 | (88.9%) | 0/11 (0%) | |
| HCV Genotype 4 | 1/1 | (100.0%) | | |
| HCV Genotype 6 | 8/8 | (100.0%) | 0/9 (0%) | |
| Others | 1/1 | (100.0%) | | |

TABLE 5
ALT normalisation at Week 12

| | IFN-beta-1a | Placebo | Overall |
|---|---|---|---|
| | N = 128 | N = 129 | N = 257 |
| Was normalisation | | | |
| achieved? | | | |
| Yes | 82 (65.1%) | 11 (8.5%) | 93 (36.5%) |
| No | 44 (34.9%) | 118 (91.5%) | 162 (63.5%) |

| Comparison | | |
|---|---|---|
| IFN-beta-1a vs placebo | | |
| Odds ratio | 20.24 | |
| 95% confidence interval | (9.833, 41.652) | |
| p-value | <0.001 | <0.001 |

Safety

No serious adverse events related to interferon-beta-1a treatment were reported in the first 12 weeks. Side effects observed were consistent with the known safety profile of interferon beta-1a administered subcutaneous at the dose of 44 mcg tiw.

Conclusion

The analysis clearly indicates that interferon-beta-1a treatment has a potent effect on viral clearance at treatment week 12. The high proportion of patients with the difficult-to-treat HCV genotype 1 infection did not seem to affect the early viral clearance rate of interferon-beta-1a treatment. Although the SVR of interferon-beta-1a treatment is yet to be confirmed by longer-term analysis, the current results indicates a possible alternative therapy for Asian patients with chronic hepatitis C infection.

### EXAMPLE 2

### Sustained Virologic Response of Interferon-Beta-1a (Rebif®) Therapy in Asian Patients with Hepatitis C: 24 and 48 Weeks Results from a Multicentre, Randomized, Placebo-Controlled Trial

Study Design and Materials and Methods:

The Study Design and the Materials And Methods employed are as in Example 1.

Results:

The Patient Demographics and the Baseline Disease Characteristics are as in Example 1.

Of the 128 subjects receiving interferon-beta-1a, 15 (11.7%) subjects withdrew before Week 48. All those subjects receiving placebo during the double-blind phase completed the first 12 weeks of the study. Two efficacy analysis populations were analysed: a Full Analysis Set and a Per Protocol Set. The Full Analysis Set was defined as all randomised and treated subjects (i.e. received at least one dose of study medication) who had at least one efficacy or safety value measured on treatment. Subjects who had missing viral load (HCV RNA) data at Week 12 were included in the Full Analysis Set as a non-responder. The Per Protocol Set was defined as all randomised and treated subjects who had no major protocol violations.

Efficacy:

Results were similar for both the Full Analysis Set and the Per Protocol Set (Table 6). In the Full Analysis Set, 26.6% (34 subjects) achieved SVR in the IFN-beta-1a group. None of the subjects randomised to placebo were responders (achieved SR).

TABLE 6
Summary of SVR at Week 48, Full Analysis Set and Per Protocol Set

| | IFN-beta-1a | Placebo | Overall | p-value |
|---|---|---|---|---|
| Number of subjects in Full Analysis Set | 128 | 129 | 257 | |
| Was SVR achieved? | | | | |
| Yes | 34 (26.6%) | 0 | 34 (13.2%) | |
| No | 94 (73.4%) | 129 (100.0%) | 223 (86.8%) | |
| 95% confidence interval | 19.1%, 35.1% | 0.0%, 2.8% | 9.3%, 18.0% | <0.001 |
| Number of subjects in Per Protocol Set | 109 | 122 | 231 | |
| Was SVR achieved? | | | | |
| Yes | 28 (25.7%) | 0 | 28 (12.1%) | |
| No | 81 (74.3%) | 122 (100.0%) | 203 (87.9%) | |
| 95% confidence interval | 17.8%, 34.9% | 0.0%, 3.0% | 8.2%, 17.0% | <0.001 |

IFN = interferon;
N = Number of subjects in the Full Analysis Set or Per Protocol Set;
SVR = sustained viral response (i.e. absence of detectable hepatitis C virus ribonucleic acid in the serum at both Week 24 and 48).
P-value from Fisher's exact test comparing SVR between placebo and IFN-beta-1a.

After evaluating SVR as a function of HCV genotype and baseline viral load, more subjects achieved SVR, regardless of genotype, when baseline viral load was low (Table 7).

TABLE 7
Summary of percentage of subjects with SVR at Week 48 as a function
of HCV genotype and baseline viral load, Full Analysis Set

| | IFN-beta-1a | | Placebo | Overall | |
|---|---|---|---|---|---|
| | N = 128 | | N = 129 | N = 257 | |
| HCV genotype | | | | | |
| All subjects | 34/128 | (26.6%) | 0/129 | 34/257 | (13.2%) |
| with SVR | | | | | |
| Genotype 1 | 19/85 | (22.4%) | 0/82 | 19/167 | (11.4%) |
| Genotype 2 or 3 | 13/33 | (39.4%) | 0/38 | 13/71 | (18.3%) |
| Genotype 4 | 1/1 | (100.0%) | 0/0 | 1/1 | (100.0%) |
| Genotype 6 | 1/8 | (12.5%) | 0/9 | 1/17 | (5.9%) |
| Genotype - other | 0/1 | | 0/0 | 0/1 | |
| Baseline HCV RNA | | | | | |
| Low viral load | 23/43 | (53.5%) | 0/47 | 23/90 | (25.6%) |
| High viral load | 11/85 | (12.9%) | 0/82 | 11/167 | (6.6%) |

HCV RNA = hepatitis C virus ribonucleic acid;
IFN = interferon:
N = number of subjects in the Full Analysis Set;
SVR = sustained viral response (i.e. absence of detectable HCV RNA in the serum at both Week 24 and 48).

Safety:

Overall, 180 (70.0%) subjects experienced at least one adverse event during the period of treatment defined as the Treatment Period plus 30 days. The majority of subjects were from the IFN-beta-1a group (123 subjects) compared to the placebo group (57 subjects). The most commonly reported events were fever (116 [45.1%] subjects), headache (59 [23.0%] subjects), fatigue (42 [16.3%] subjects) and injection site rash (41 [16.0%] subjects). The majority were reported by subjects from the IFN-beta-1a group and were events previously associated with IFN treatment. There were no deaths reported during the study. One subject receiving placebo reported an SAE of a perforated gastric ulcer. Four subjects discontinued study medication as a result of an adverse event (leucopenia, injection site reaction, fever and arthralgia, and thrombocytopenia). Mean values for platelets in the IFN-beta treated group fell below the normal reference range reaching its lowest value at Week 4 before climbing back within a normal range by Week 20 of treatment. Although decreased in the IFN-beta-1a group, mean values for haemoglobin, WBC and neutrophils (absolute) remained within the normal reference range at all times. There was one subject with a grade 4 toxicity for abnormal neutrophils from the placebo group. No other Grade 4 toxicities were recorded. There were no abnormalities of note in vital signs. A summary of adverse events (AE) is given in Table 8 below.

TABLE 8
Summary of adverse events during the period of treatment

| | IFN-beta-1a | Placebo | Overall |
|---|---|---|---|
| Number of subjects, | 128 | 129 | 257 |
| Safety Set | | | |
| Number of adverse | 611 | 139 | 750 |
| events | | | |
| Number of subjects | | | |
| with at least one: | | | |
| Adverse event | 123 (96.1%) | 57 (44.2%) | 180 (70.0%) |
| Serious adverse event^{A} | 0 | 1 (0.8%) | 1 (0.4%) |
| Adverse event leading | 3 (2.3%) | 1 (0.8%) | 4 (1.6%) |
| to discontinuation | | | |
| of study medication | | | |
| Adverse event | 123 (96.1%) | 40 (31.0%) | 163 (63.4%) |
| considered probably or | | | |
| possibly related to | | | |
| study medication | | | |

Conclusions:
The primary efficacy endpoint for the proportion of subjects with SVR (the absence of detectable HCV RNA in the serum at Week 24 and Week 48) was met.
There was a statistically significant difference (p value <0.001) between the proportion of subjects with SVR in the IFN beta 1a group (26.6%) compared with the placebo group (0%).
In determining the sample size, an outcome of 15% of subjects with SVR was considered “clinically acceptable and worthwhile”. These data exceed that outcome.
The most commonly reported adverse events included fever, headache, fatigue and injection site reactions; there were no new or unexpected events observed.

IFN-beta-1a as monotherapy for subjects with chronic hepatitis C was effective with no safety concerns.

### EXAMPLE 3

### Sustained Virologic Response of Interferon-Beta-1a (Rebif®) And Ribavirin Combination Therapy in Asian Patients with Hepatitis C: 24 and 48 Weeks Results from a Multicentre, Randomized, Placebo-Controlled Trial

An analysis of the final trial data has been performed and the results of the IFN-beta and ribavirin combination treatment group from the draft statistical report are presented below.

TABLE 9
Summary of SVR at Week 48, Full Analysis Set

| | | IFN-beta-1a | |
|---|---|---|---|
| | IFN-beta-1a | & Ribavirin | Overall |
| Number of Patients | 128 | 127 | 255 |
| Was SVR Achieved? | | | |
| Yes | 34 (26.6%) | 73 (57.5%) | 107 (42.0%) |
| No | 94 (73.4%) | 54 (42.5%) | 148 (58.0%) |
| 95% Confidence Interval | [19.1%, | [48.4%, | [35.8%, |
| | 35.1%] | 66.2%] | 48.3%] |

### EXAMPLE 3A

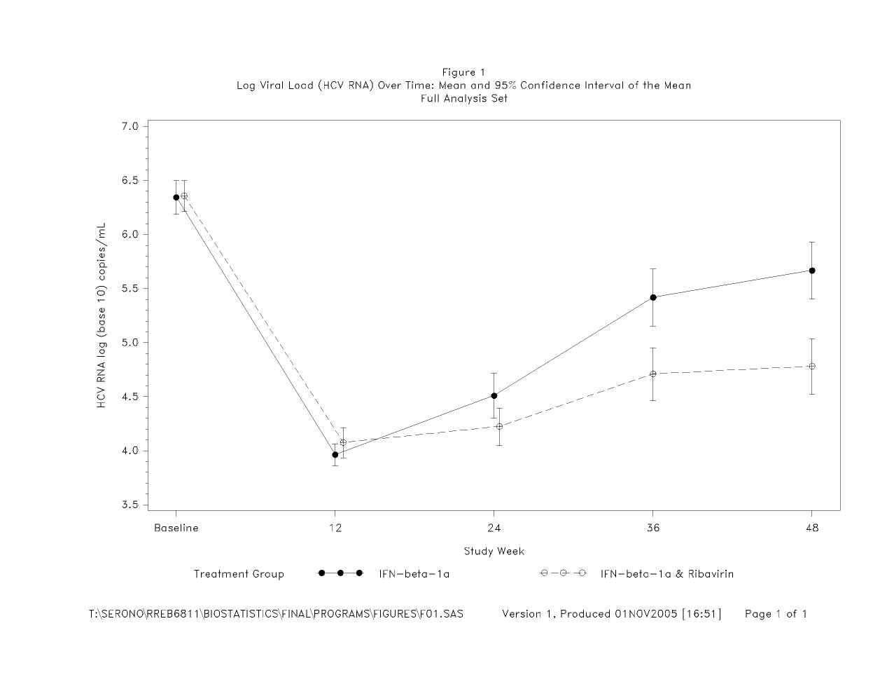

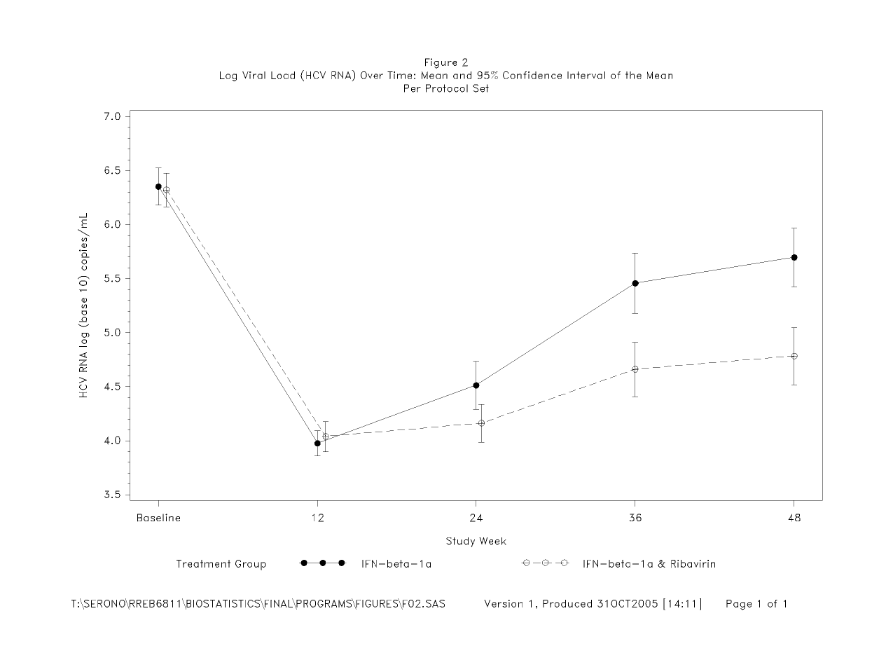

### Viral Load (FIGS. 1 and 2)—Monotherapy and Combination Therapy with Ribavirin

The viral load (quantity of virus) in the blood at 5 time points (baseline, week 12, 24, 36 & 48) is measured during the study (FIGS. 1 and 2). The mean (average) viral load for all the patients in each treatment arms (monotherapy and combotherapy) is given. A quantitative HCV RNA test is performed. As the test has a detection limit (cannot detect the virus if the quantity is below certain level), the viral load is given at 3.8 log 10 copies/ml (detection limit).

### EXAMPLE 3B

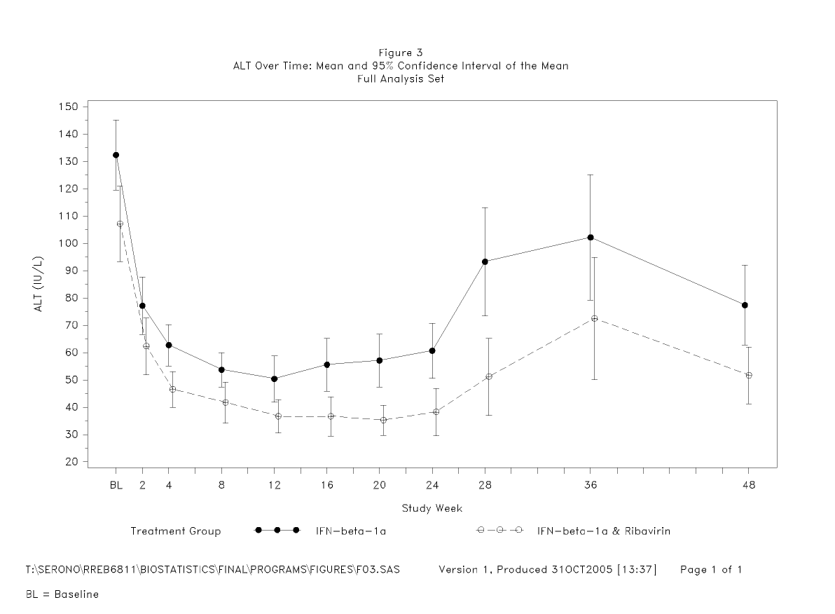

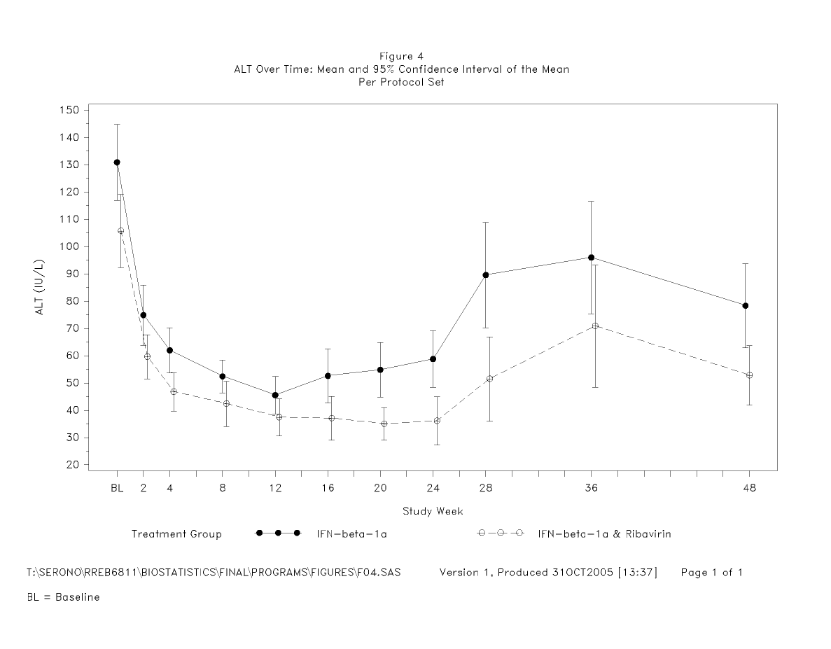

### ALT levels (FIGS. 3 and 4)—Monotherapy and Combination Therapy with Ribavirin

The ALT (liver enzyme) levels in the blood at 11 time points are measured during the study (FIGS. 3 and 4). The mean (average) level is given for all the patients in each treatment arms (monotherapy and combotherapy). ALT is considered in the normal range when it drops below 48 IU/L.

### REFERENCES

Alter M J et al., N. Engl. J. Med., 327, 1899-1905, 1992;
Altschul S F et al, J Mol Biol, 215, 403-410, 1990
Altschul S F et al, Nucleic Acids Res., 25:389-3402, 1997;
Ansaldi et al., J. Med. Virol. 2001, 63:17-21.
Bacon B R et al., Hepatology, 22, 152A-152A, 1995;
Bonkovsky H L. et al., Hepatology, 25(3), 759-768, 1997;
Brunt E M, Hepatology 2000; 31 (1); 241-246)
Cavalli-Sforza L, Scientific American, 72-78, Nov. 1991
Conjeevaram H S et al., Hepatology, 22(4 Pt 1), 1326-1329. 1995;
Davis G L, J. Hepatol., 11, S72-S77, 1990;
Davis G L et al., Hepatology 2003, 38(3), 645-652.
Derynk R. et al., Nature 285, 542-547, 1980;
Devereux J et al, Nucleic Acids Res, 12, 387-395, 1984;
Grantham et al., Science, Vol. 185, pp. 862-864 (1974)
Habersetzer et al., Liver, 20 437-441, 2000;
Hoofnagle J H, N. Engl. J. Med., 336 (5):347-356, 1997;
Kishiara et al., Fukukoka Acta Med., 86(4), 113-20, 1995;
Lindsay K L, Hepatology, 24, 1034-1040, 1996;
Mark D. F. et al., Proc. Natl. Acad. Sci. U.S.A., 81 (18) 5662-5666, 1984;
Omata M et al., Lancet, 338, 914-915, 1991;
Pearson W R, Methods in Enzymology, 183, 63-99, 1990
Pearson W R and Lipman D J, Proc Nat Acad Sci USA, 85, 2444-2448, 1988
Perez R. et al., J. Virol. Hepat., 2(2), 103-6), 1995;
Piccinino F., Arch. Virol. Suppl., 8, 257-263, 1993;
Poynard T. et al., Hepatology, 24, 778-789, 1996;
Ross et al., J. Clin. Microbiol. 2000, 38:3581-3584.
Schvarcz R., Scand. J. Infect. Dis., 21, 617-625, 1989;
Shepard H. M. et al., Nature, 294, 563-565, 1981;
Sievert, J. Gast. Hep. 2002, 17: 415-422.
Simmonds P., J. Hepatol. 1999, 31(suppl. 1): 54-60.
Strader et al., Hepatology 2004, 39(4), 1147-1171.
Stuyver et al., J. Clin. Microbiol., 1996, 34: 2259-2266.
Tabor E et al, J. Natl. Cancer Inst., 84 (2), 86-90, 1992;
Takeda T., Gastroenterol Jpn., 28, 104-108, 1993;
Wilkinson T, Curr. Op. Invst. Drug, 2(11), 1516-22, 2002
Youcefi et al., Am J Clin Pathol. 1985 June; 83(6):735-40
WO 97/24137
WO 99/55377
WO 00/23472
WO 01/03737
EP 526 452
EP 227 110
U.S. Pat. No. 4,738,931
U.S. Pat. No. 4,959,314
U.S. Pat. No. 4,588,585
U.S. Pat. No. 5,017,691
U.S. Pat. No. 4,879,111
U.S. Pat. No. 4,695,195
U.S. Pat. No. 5,116,943
U.S. Pat. No. 4,904,584
U.S. Pat. No. 6,013,253

1-16. (canceled)

17. A method of treatment of a interferon-α-naïve patient belonging to the Asian population with a hepatitis C virus (HCV) infection, comprising administering to the patient an effective amount of interferon-β (IFN-β), or a mutein, functional derivative, active fragment or fusion protein thereof, having IFN-β activity.

18. The method according to claim 17, wherein the patient has not been treated with a combination of IFN-α and ribavirin.

19. The method according to claim 17, wherein the HCV infection is a chronic HCV infection.

20. The method according to claim 17, wherein the HCV is selected from genotype 1, 4, 5 or 6.

21. The method according to claim 17, wherein the patient has a high viral load of over 2×10⁶ copies/ml.

22. The method according to claim 17, wherein the patient has a sustained viral response.

23. The use according to any of the preceding claims, wherein IFN-β is recombinant IFN-β.

24. The method according to claim 17, wherein the IFN-β is IFN-β-1a.

25. The method according to claim 17, wherein the IFN-β functional derivative comprises one or more polyethyleneglycol (PEG) moieties.

26. The method according to claim 17, wherein the IFN-β fusion protein is an immunoglobulin fusion protein.

27. The method according to claim 17, wherein the IFN-β is administered subcutaneously.

28. The method according to claim 17, wherein dosages and regimens of the treatment are selected from the group consisting of: 12 MIU (44 mcg) three times a week, 12 MIU (44 mcg) daily, 24 MIU (88 mcg) three times a week, and 24 MIU (88 mcg) daily.

29. The method according to claim 17, further comprising treating the patient with ribavirin.

30. The method according to claim 29, wherein the ribarivin is administered orally.

31. The method according to claim 30, wherein the ribavirin is administered at about 800 to 1400 mg per person per day.

32. A method of treatment of a interferon-α-naïve patient belonging to the Asian population with a hepatitis C virus (HCV) infection, comprising administering to the patient an effective amount of a pharmaceutical composition comprising interferon-β (IFN-β), or a mutein, functional derivative, active fragment or fusion protein thereof, having IFN-β activity, together with a pharmaceutically acceptable excipient.
